(19) United States
(12) Patent Application Publication
(10) Pub. No.: US 2011/0262457 A1
(43) Pub. Date: Oct. 27, 2011

(54) MODULATION OF THE IMMUNE RESPONSE

(76) Inventors: Howard Weiner, Brookline, MA (US); Francisco J. Quintana, Jamaica Plain, MA (US)

(21) Appl. No.: 12/933,800

(22) PCT Filed: Mar. 19, 2009
(86) PCT No.: PCT/US09/37696

§ 371 Date: Jul. 7, 2011

Related U.S. Application Data
(60) Provisional application No. 61/070,410, filed on Mar. 21, 2008.

(51) Int. Cl. A61K 31/407; A61K 35/26; A61K 39/395; A61K 39/00; A61K 39/12; A61K 39/02; A61K 39/002; A61P 37/02; A61P 35/00; A61P 31/12; A61P 31/04; A61P 31/10; A61P 33/02; A61K 31/352; C12N 5/00

(52) U.S. Cl. 424/172.1; 435/375; 424/93.7; 514/410; 424/178.1; 424/184.1; 424/204.1; 424/234.1; 424/274.1; 424/269.1; 514/455

(57) ABSTRACT

Methods for increasing the generation of IL-17-producing T cells (T_{H}17) in vivo and in vitro, and enriched populations of T_{H}17 cells for the treatment of diseases benefiting from an induced or enhanced immune response, e.g., infection and cancer.

### CLAIM OF PRIORITY

This application claims the benefit of U.S. Provisional Patent Application Ser. No. 61/070,410, filed on Mar. 21, 2008, and of International Patent Application No. PCT/US2008/083016, filed on Nov. 10, 2008, the entire contents of which are hereby incorporated by reference.

### FEDERALLY SPONSORED RESEARCH OR DEVELOPMENT

This invention was made with Government support under Grant Nos. AI435801, AI043458, and NS38037 awarded by the National Institutes of Health. The Government has certain rights in the invention.

### TECHNICAL FIELD

This invention relates to methods and compositions for enhancing a subject's immune response by increasing the number and/or activity of IL-17-producing T cells (T_{H}17)) in vivo and in vitro.

### BACKGROUND

Regulatory T cells (Treg) control the autoreactive components of the immune system. The development of Treg is reciprocally related to that of proinflammatory T cells producing IL-17 (T_{H}17). T_{H}17 cells express the transcription factor retinoic acid-related orphan receptor gamma t (RORγt) (Ivanov et al., Nat. Immunol., 8:345-50, 2007), participate in the control of extracellular pathogens and play an important role in human and experimental autoimmunity (Bettelli et al., Nat. Immunol., 8:345-350, 2007; O'Quinn and Palmer, Adv. Immunol., 99:115-163 (2008)).

Although both cell types are thought to contribute to various immunological conditions, little is know about the physiological pathways or mechanisms that lead to the generation and/or activation of these cell types. For example, TGFβ1 induces the differentiation of Treg (Chen et al., J. Exp. Med., 198:1875-1886, 2003). In contrast, TGFβ1 in combination with IL-6 (Veldhoen et al., Immunity, 24:179-189, 2006) or IL-21 (Korn et al., Nature, 448:484-7, 2007) results in the differentiation of T_{H}17 cells.

Because of the central role Treg and T_{H}17 play in the immune response to pathogens, characterization of the pathways involved and identification of compounds capable of modulating these pathways, e.g., to promote the generation (e.g., differentiation of cells to or towards) of T_{H}17 cells or to promote increased activity of T_{H}17 cells is important for the treatment of, e.g., infections and cancer.

### SUMMARY

The present invention is based, at least in part, on the discovery that compounds capable of modulating (e.g., increasing or decreasing) the expression and/or activity of the Aryl Hydrocarbon Receptor (AHR) provide useful targets for enhancing the immune response. AHR activation by 2,3,7,8-tetrachlorodiberizo-p-dioxin (TCDD) induced Treg cells that suppressed experimental autoimmune encephalomyelitis (EAE) by a TGT-b1-dependent mechanism, whereas AHR activation by 6-formylindolo[3,2-b]carbazole (FICZ) or beta-naphthoflavone (bNF) interfered with Treg cell differentiation, boosted T_{H}17 cell differentiation and worsened EAE. Thus, AHR regulates Treg and T_{H}17 cell differentiation in a ligand-specific manner. Accordingly, the present invention provides, inter alia, compositions and methods for the prevention or treatment of diseases caused by a deficient (e.g., absent or insufficient) immune response.

In one aspect, the invention features methods for increasing the number or activity of T cells producing IL-17 (T_{H}17) in a population of T cells. The methods include contacting the population of cells with a sufficient amount of a composition comprising an AHR ligand that reduces expression of Foxp3, e.g., 6-formylindolo[3,2-b]carbazole (FICZ) or beta-naphthoflavone (bNF), optionally linked to a biocompatible nanoparticle, and optionally evaluating the presence and/or number of IL-17-expressing cells in the population. The methods result in an increase in the number and/or activity of T_{H}17 cells.

In some embodiments, the initial population of T cells includes one or both of naïve T cells or CD4⁺CD62 ligand⁺ T cells. The population of T cells can be isolated, i.e., in vitro, or in a living mammalian subject, e.g., a subject who has a tumor, an infection, or is immunosuppressed. The population of cells can also be administered as an adjuvant, e.g., in young children or the elderly, to boost the immune response to a vaccine. In particular, the present methods can be used in subjects who are immunosuppressed as a result of infection with human immunodeficiency virus (HIV), a condition that is often associated with a deficit of T_{H}17 cells. In embodiments where the T cells are in a living subject, the methods can include administering the one or more ligands orally, mucosally, or intravenously.

In some embodiments, T_{H}17 cells generated or activated using a method described herein are administered to a subject who is suffering from a tumor or an infection, or who is immunosuppressed, in an amount sufficient to improve or ameliorate a symptom of the disorder.

Also provided herein are methods for identifying candidate compounds that increase generation or activity of T_{H}17 cells. The methods include providing a cell expressing a reporter construct comprising a binding sequence for the Aryl Hyrocarbon Receptor (AHR) in a mammalian Foxp3 promoter sequence, wherein said binding sequence is operably linked to a reporter gene, for example a reporter gene selected from the group consisting of luciferase, green fluorescent protein, and variants thereof; contacting the cell with a test compound; and evaluating an effect of the test compound on expression of the reporter gene. A test compound that increases or decreases expression of the reporter gene is a candidate compound that modulates generation of T_{H}17 cells.

The methods can optionally include measuring expression of the reporter construct in the presence of an AHR ligand that reduces expression of Foxp3, e.g., FICZ or bNF; determining whether the candidate compound competes for binding to the AHR with the known compound; and selecting the candidate compound if it binds the AHR competitively with the known compound.

In a further aspect, the present invention provides methods of identifying candidate compounds that modulate the generation of T cells producing IL-17 (T_{H}17). These methods include providing a cell expressing a reporter construct containing a binding sequence for AHR operably linked to a reporter gene. The cell is then contacted with a test compound, and the effect of the test compound on expression of the reporter gene is evaluated. A test compound that increases or decreases expression of the reporter gene is a candidate compound that modulates generation of T_{H}17 cells.

In another aspect, the present invention provides methods of identifying candidate compounds that modulate generation of T_{H}17 cells. These methods include providing a living zebrafish, e.g., a zebrafish embryo, e.g., 30 minutes after the egg is laid; contacting the zebrafish with a test compound, e.g., by putting the test compound in water in which the zebrafish is living or microinjecting the compound into an embryo; and evaluating an effect of the test compound on Foxp3 expression in the zebrafish, wherein a test compound that increases or decreases expression of Fox-3 in the zebrafish is a candidate compound that modulates generation of T_{H}17 cells.

In a further aspect, the present invention provides compositions comprising transcription factor ligands capable of promoting increased expression, activity, or both of a Foxp3 gene.

In yet another aspect, the present invention provides methods for increasing the numbers of T_{H}17 cells in a population of T cells. These methods include contacting the cell with one or more AHR ligands that reduce expression of Foxp3, e.g., 6-formylindolo[3,2-b]carbazole (FICZ) or beta-naphthoflavone (bNF), wherein the method results in an increase in the number and/or activity of regulatory IL-17-producing T cells (T_{H}17).

In an additional aspect, the present invention provides methods for increasing the numbers of T_{H}17 cells in a subject. These methods include administering one or more AHR ligands to a subject selected for treatment, e.g., 6-formylindolo[3,2-b]carbazole (FICZ) or beta-naphthoflavone (bNF), wherein the method results in an increase in the number and/or activity of IL-17-producing T cells (T_{H}17).

In another aspect, the invention provides methods for preparing an enriched population of T cells producing IL-17 (T_{H}17) from an initial population of T cells. The methods include providing an initial population of T cells; contacting the population of cells with a sufficient amount of a composition comprising an AHR ligand, e.g., 6-formylindolo[3,2-b]carbazole (FICZ) or beta-naphthoflavone (bNF), and optionally evaluating the presence and/or number of T_{H}17 cells in the population. The method results in an increase in the number of regulatory T_{H}17 cells in the population.

In some embodiments, the initial population of T cells includes naïve T cells and/or CD4⁺CD62 ligand⁺ T cells.

In some embodiments, the population of T cells is in vitro, and the methods further include contacting the cells with an effective amount of one or both of interleukin-6 (IL-6) and transforming growth factor (TGF)-beta.

In some embodiments, the methods further include contacting the cells with one or more antibodies that selectively bind to an antigen present on a T cell, a B cell, a dendritic cell, or a macrophage. In some embodiments, the antibody is linked to a biocompatible nanoparticle. In embodiments where both the antibody and the AHR ligand are linked to nanoparticles, they can be present on the same nanoparticles or on separate nanoparticles.

In some embodiments, the methods further include preparing the enriched population for administration to a subject. In some embodiments, the methods further include administering the T_{H}17 cells to a subject suffering from a disorder that would benefit from an enhanced T_{H}17-mediated immune response, in an amount sufficient to improve or ameliorate a symptom of the disorder.

In another aspect, the invention features methods for treating a subject having a disease that would benefit from an enhanced T_{H}17-mediated immune response, e.g., a tumor or an infection with a pathogen, e.g., a virus, fungus, bacterium, or protozoa. The methods include identifying a subject in need of treatment that would increase an immune response, e.g., selecting a subject on the basis that they have a disease that would benefit from increased immune response; and administering to the subject a composition comprising a therapeutically effective amount of an AHR ligand, e.g., 6-formylindolo[3,2-b]carbazole (FICZ) or beta-naphthoflavone (bNF), thereby treating the subject.

In some embodiments, the subject is infected with a pathogen selected from the group consisting of viruses, bacteria, fungi, and protozoa. In some embodiments, the subject has cancer.

In some embodiments, the FICZ or bNF is linked to a biocompatible nanoparticle.

In some embodiments, the methods further include administering to the subject one or more antibodies that selectively bind to an antigen present on a T cell, a B cell, a dendritic cell, or a macrophage. In some embodiments, the antibody is linked to a biocompatible nanoparticle. In embodiments where both the antibody and the AHR ligand are linked to nanoparticles, they can be present on the same nanoparticles or on separate nanoparticles.

In some embodiments, the methods include administering an antigen associated with the disease in the subject, e.g., a tumor-associated antigen or an antigen that is associated with a pathogen selected from the group consisting of viruses, bacteria, fungi, and protozoa, depending on which pathogen the subject is infected with.

In one aspect, the present invention features compositions including a ligand that binds specifically to an aryl hydrocarbon receptor (AHR) transcription and that reduces expression of Foxp3, e.g., linked to a biocompatible nanoparticle. The ligand can be, e.g., 6-formylindolo[3,2-b]carbazole (FICZ) or beta-naphthoflavone (bNF).

In some embodiments, the composition also includes an antibody that selectively binds to an antigen present on a T cell, a B cell, a dendritic cell, or a macrophage. The antibody can be present on (i.e., linked to) the same nanoparticles, linked to different nanoparticles (of the same or different types) or free in solution.

In some embodiments, the composition also includes an inhibitor of degradation of the ligand, e.g., a monoamine oxidase inhibitor such as tranylcypromine. The inhibitor can be present on (i.e., linked to) the same nanoparticles, linked to different nanoparticles (of the same or different types) or free in solution. In some embodiments, the methods and compositions described herein include the use of a ligand that binds specifically to an aryl hydrocarbon receptor (AHR) transcription factor and reduces expression of Foxp3, e.g., and an inhibitor of degradation thereof, wherein both, one, or neither is linked to a nanoparticle.

As used herein, “treatment” means any manner in which one or more of the symptoms of a disease or disorder are ameliorated or otherwise beneficially altered. As used herein, amelioration of the symptoms of a particular disorder refers to any lessening, whether permanent or temporary, lasting or transient of the symptoms, that can be attributed to or associated with treatment by the compositions and methods of the present invention.

The terms “effective amount” and “effective to treat,” as used herein, refer to an amount or a concentration of one or more of the compositions described herein utilized for a period of time (including acute or chronic administration and periodic or continuous administration) that is effective within the context of its administration for causing an intended effect or physiological outcome.

The term “subject” is used throughout the specification to describe an animal, human or non-human, rodent or non-rodent, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

The term gene, as used herein refers to an isolated or purified gene. The terms “isolated” or “purified,” when applied to a nucleic acid molecule or gene, includes nucleic acid molecules that are separated from other materials, including other nucleic acids, which are present in the natural source of the nucleic acid molecule. An “isolated” nucleic acid molecule, such as an mRNA or cDNA molecule, can be substantially free of other cellular material, or culture medium when produced by recombinant techniques, or substantially free of chemical precursors or other chemicals when chemically synthesized.

An “isolated” or “purified” polypeptide, peptide, or protein is substantially free of cellular material or other contaminating proteins from the cell or tissue source from which the protein is derived, or substantially free from chemical precursors or other chemicals when chemically synthesized. “Substantially free” means that the preparation of a selected protein has less than about 30%, (e.g., less than 20%, 10%, or 5%) by dry weight, of non-selected protein or of chemical precursors. Such a non-selected protein is also referred to herein as “contaminating protein”. When the isolated therapeutic proteins, peptides, or polypeptides are recombinantly produced, it can be substantially free of culture medium, i.e., culture medium represents less than about 20%, (e.g., less than about 10% or 5%) of the volume of the protein preparation. The invention includes isolated or purified preparations of at least 0.01, 0.1, 1.0, and 10 milligrams in dry weight.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1I show that treatment of T cells with AHR ligands induced differentiation into either Treg or T_{H}17, depending on the ligand used. FIG. 1A is a set of three FACS plots showing conversion of CD4⁺Foxp3:GFP⁻ T cells into CD4⁺Foxp3:GFP⁺ Treg by stimulation with antibodies to CD3 and CD28 in the presence of TGFb1 with or without FICZ; FIG. 1b; FIG. 1B presents the data in bar graph format. FIG. 1C is a bar graph showing AHR expression in naïve T cells differentiated in vitro into T_{H}1, T_{H}2 or T_{H}17 cells for four days (mean+s.d. of triplicates normalized to actin expression). FIG. 1D is a bar graph showing AHR expression in naïve T cells differentiated in vitro into T_{H}17 for four days with the indicated cytokines (mean+s.d. of triplicates normalized to actin expression). FIG. 1E is a bar graph showing RORγt expression in naïve T cells differentiated into T_{H}17 for four days with TGFβ1 and IL-6 alone or in combination with FICZ (mean+s.d. of triplicates normalized to actin expression). FIG. 1F is a set of nine FACS plots of frequency of IL-17⁺ T cells differentiated with TGFβ1 and IL-6 alone or in combination with IL-23 and/or FICZ for four days. FIG. 1G is a bar graph showing IL-17 and FIG. 1H is a pair of bar graphs showing IL-21 (left) and IL-22 (right) levels in supernatants of cultures prepared as in 1E, as measured by ELISA after 4 days of differentiation into. FIG. 1I is a bar graph showing Inhibition of T_{H}17 differentiation by the AHR antagonist resveratrol.

FIG. 2A is a line graph showing development of EAE on FICZ⁻ or control-treated mice (mean EAE score+s.e.m.)

FIG. 2B is a pair of bar graphs showing cytokine secretion (pg/ml) triggered by MOG₃₅₋₅₅ in splenocytes taken from FICZ or control-treated mice; left, IFNgamma, right, IL-17.

FIGS. 2C-2E are each pairs of FACS plots showing the frequency of IFNγ, (2C), IL-17 (2D) or Foxp3 (2E) in splenocytes from FICZ or control-treated mice.

FIGS. 3A and 3B are bar graphs illustrating AFP levels in HCC tumor models treated with FICZ (3A) or FICZ plus HBSAg (3B).

FIGS. 4A-4C are bar graphs showing changes in levels of CD3 (4A), IFNgamma (4B), and IL-17 (4C) in a zebrafish model of EAE.

FIG. 4D is a bar graph showing the effect of a specific morpholino antisense oligonucleotide against Foxp3 on IL-17 expression in zebrafish.

FIG. 5 is a schematic illustration of gold nanoparticles for AHR-ligand delivery.

FIGS. 6A and 6B show the functionality of gold nanoparticles containing AHR-ligands. 6A is a bar graph showing activation of luciferase activity in an AHR reporter cell line by nanoparticles linked to AHR ligands TCDD and FICZ. 6B is the absorption spectra of the nanoparticles constructed.

FIG. 7 is a line graph showing modulation of EAE by AHR-ligand nanoparticles. EAE was induced in B6 mice (n=5), the mice were treated with nanoparticles weekly starting from day 0, and the animals were followed for signs of EAE.

FIG. 8 is a bar graph showing fluorescence in 293 cells transfected with an AHR reporter luciferase construct and a TK-Renilla Luciferase construct for normalization purposes. The cells were incubated with different concentrations of the AHR ligand TCDD and activation of the AHR reporter was followed by monitoring fluorescence from the luciferase.

FIG. 9 is a bar graph T cells differentiation into Th17 cells by in vitro activation with antibodies to CD3 and CD28 in the presence of TGF-beta and IL-21, in the presence of or not of showing beta-naphthoflavone (bNF) or FICZ (100 nM). IL-17 production was measured by real time PCR.

### DETAILED DESCRIPTION

Because of the importance of the central role T cells producing IL-17 (T_{H}17) play in inflammation and the immune response to tumors and pathogens, characterization of the pathways and identification of compounds capable of modulating these pathways, e.g., to promote the generation (e.g., differentiation of cells to or towards) T_{H}17 cells or that promote increased activity of T_{H}17 cells is important for the treatment of, e.g., infections and cancer.

The present invention provides, inter alia, compositions and methods useful for therapeutic immunomodulation.

Accordingly, the present invention is based, at least in part, on the discovery that modulation of the AhR by certain high-affinity ligands as described herein can be used to modulate (e.g., increase or decrease the number and/or activity of) T_{H}17 cells in vitro and in vivo. Interestingly, other AHR-specific ligands such as T also regulate differentiation of regulatory T cells (Treg) (see PCT International Patent Application NO. PCT/US2008/083016, and U.S. Provisional Patent Application Ser. No. 60/989,309, filed on Nov. 20, 2007, both of which are incorporated herein by reference in their entirety), making the AHR a useful target for immune-based therapies.

In some embodiments, the present invention is based on the identification of the ligand-activated transcription factor aryl hydrocarbon receptor (AHR) as a regulator of T_{H}17 differentiation (e.g., generation) and/or activity in vitro and in vivo. Also described herein are ligands of AHR that are useful for promoting the differentiation and/or activation of T_{H}17 cells. More specifically, the data presented herein demonstrates the use of AHR-specific ligands that reduce expression of Foxp3, e.g., 6-formylindolo[3,2-b]carbazole (FICZ) or beta-naphthoflavone (bNF), to promote an increase in the number and/or activity of T cells producing IL-17 (T_{H}17), which will be useful to suppress the immune response in the treatment of diseases or disorders associated with an abnormally low immune response, or disorders that would benefit from an enhanced immune response, e.g., infections or cancer. In some embodiments, effective doses of the ligand, e.g., FICZ or bNF, can be administered intravenously or orally.

The data presented herein demonstrates the use of AHR ligands that reduce expression of Foxp3, e.g., 6-formylindolo[3,2-b]carbazole (FICZ) or beta-naphthoflavone (bNF), to promote an increase in the number and/or activity of T_{H}17 immunomodulatory cells, which will be useful to enhance or promote the immune response in the treatment of diseases or disorders caused by an absent or insufficient immune response (e.g., cancer and infection).

Compounds that Increase Generation and/or Activity of T_{H}17

As described herein, AHR ligands that reduce expression of Foxp3, e.g., FICZ or bNF, increase the generation and/or activity of T_{H}17 cells and is therefore useful in methods of enhancing immune response. Other compounds that act on the AHR as FICZ or bNF does can also be used; a number of other compounds that bind to the AHR are known, and simple assays can be used to determine whether they also increase generation and/or activity of T_{H}17 cells.

In some embodiments of the methods described herein, a composition including an AHR ligand that reduces expression of Foxp3, e.g., 6-formylindolo[3,2-b]carbazole (FICZ) or beta-naphthoflavone (bNF), is administered to a subject in vivo or to a population of cells in vitro. In some embodiments, the ligand is linked to a biocompatible nanoparticle.

In some embodiments, the composition also includes an antibody that selectively binds to an antigen present on a T cell, a B cell, a dendritic cell, or a macrophage. The antibody can be present on (i.e., linked to) the same nanoparticles, linked to different nanoparticles (of the same or different types) or free in solution.

In some embodiments, e.g., when the ligand is administered in vivo, the composition also includes a specific antigen, to induce an antigen-specific response. The antigen can be, e.g., a tumor- or pathogen-specific antigen.

AHR

Exemplary human AHR mRNA sequences are known in the art and include Genbank Acc. No. NM_{—}001621.3; the amino acid sequence of the protein is Genbank Acc. No. NP_{—}001612.1. Active fragments of AHR are DNA binding fragments with transcription activity, and contain at least one PAS region, e.g., amino acids 122-224 or 282-381 of NP_{—}001612.1. Consensus recognition sequences that bind AHR include the sequence TNGCGTG.

In some embodiments, the assays include the use of nucleic acids or polypeptides that are at least 80% identical to a human AHR sequence, e.g., at least 80%, 85%, 90%, or 95% identical to a human sequence as described herein.

To determine the percent identity of two sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In the present methods, the percent identity between two amino acid sequences is determined using the Needleman and Wunsch ((1970) J. Mol. Biol. 48:444-453) algorithm which has been incorporated into the GAP program in the GCG software package (available on the world wide web at gcg.com), using a Blossum 62 scoring matrix with a gap penalty of 12, a gap extend penalty of 4, and a frameshift gap penalty of 5.

Test Compounds

Test compounds for use in the methods described herein are not limited and can include crude or partially or substantially purified extracts of organic sources, e.g., botanical (e.g., herbal) and algal extracts, inorganic elements or compounds, as well as partially or substantially purified or synthetic compounds, e.g., small molecules, polypeptides, antibodies, and polynucleotides, and libraries thereof.

A test compound that has been screened by a method described herein and determined to increase levels and/or activity of T_{H}17 cells herein can be considered a candidate compound for the treatment of a disorder that would benefit from an enhanced immune response, e.g., cancer or an infection. A candidate compound that has been screened, e.g., in an in vivo model of such a disorder, e.g., cancer or an infection, and determined to have a desirable effect on the disorder, e.g., on one or more symptoms of the disorder, can be considered a candidate therapeutic agent. Candidate therapeutic agents, once screened and verified in a clinical setting, are therapeutic agents. Candidate therapeutic agents and therapeutic agents can be optionally optimized and/or derivatized, and formulated with physiologically acceptable excipients to form pharmaceutical compositions.

In some embodiments the test compounds are known to bind the AHR. AHR transcription factor ligands are described in Denison and Nagy, Ann. Rev. Pharmacol. Toxicol., 43:309-34, 2003, and references cited herein, all of which are incorporated herein in their entirety. Other such molecules include planar, hydrophobic HAHs (such as the polyhalogenated dibenzo-pdioxins, dibenzofurans, and biphenyls) and PAHs (such as 3-methylcholanthrene, benzo(a)pyrene, benzanthracenes, and benzoflavones), and related compounds. (Denison and Nagy, 2003, supra). Nagy et al., Toxicol. Sci. 65:200-10 (2002), described a high-throughput screen useful for identifying and confirming other ligands. See also Nagy et al., Biochem. 41:861-68 (2002). In some embodiments, those ligands useful in the present invention are those that bind competitively with FICZ or bNF.

Methods of Identifying Therapeutic Compounds

A number of methods are known in the art for evaluating whether a compound increases generation and/or activity of T_{H}17 cells. For example, in some embodiments a compound that is useful in the methods described herein binds to the AHR, e.g., competes for binding of the AHR with FICZ or bNF, and thereby increases generation and/or activity of T_{H}17 cells. In some embodiments, suitable compounds will also result in an increase in levels of RORγt, a transcription factor associated with differentiation of T_{H}17 cells. In some embodiments, suitable compounds will also result in an increase in levels of IL-17.

Methods of assessing binding are known in the art, see, e.g., Goodrich and Kugel, Binding and Kinetics for Molecular Biologists (Cold Spring Harbor Laboratory Press; 1st edition (Nov. 30, 2007)); and Odell and Franchimont, Principles of Competitive Protein Binding Assays (John Wiley & Sons Inc; 2nd edition (November 1982)). Methods of assessing mRNA levels are well known in the art and include, but are not limited to, Northern analysis, ribonuclease protection assay, reverse transcription-polymerase chain reaction (RT-PCR), real time PCR, and RNA in situ hybridization (see, e.g., Sambrook et al., Molecular Cloning: A Laboratory Manual, 3^{rd} Ed., Cold Spring Harbor Laboratory Press (2001)). Levels of proteins and peptides can be monitored by, e.g., Western analysis, immunoassay, in situ hybridization, or intracellular staining/FACS analysis (see, e.g., Ivanov et al., Nat. Immunol., 8:345-50, 2007). Transcription factor activity, e.g., altered promoter binding and/or transcription activity, can be determined by, e.g., electrophoretic mobility shift assay, DNA footprinting, reporter gene assay, or a serine, threonine, or tyrosine phosphorylation assay. In some embodiments, the effect of a test compound on expression, level or activity is observed as a change in glucose tolerance or insulin secretion of the cell, cell extract, co-culture, explant, or subject. In some embodiments, the effect of a test compound on expression, level, or activity of a transcription factor is evaluated in a transgenic cell or non-human animal, or explant, tissue, or cell derived therefrom, having altered glucose tolerance or insulin secretion, and can be compared to a control, e.g., wild-type animal, or explant or cell derived therefrom.

The effect of a test compound on expression, level, or activity can be evaluated in a cell, e.g., a cultured mammalian cell, a primary cell, cell lysate, or subject, e.g., a non-human experimental mammal such as a rodent, e.g., a rat, mouse, or rabbit, or a cell, tissue, or organ explant, e.g., pancreas or pancreatic cells.

In some embodiments, the ability of a test compound to increase generation and/or activity of T_{H}17 cells is further evaluated in an animal, e.g., an experimental animal. In these methods, a compound identified by an in vitro method described herein is administered to an animal for validation. Levels of T_{H}17 cells can be determined using known methods. Alternatively or in addition, levels of IL-17, IL-21, or IL-22 can also be evaluated, e.g., using ELISA, ELISPOT, or RT-PCR assays as known in the art (see, e.g., O'Quinn and Palmer, Adv. Immunol., 99:115-163 (2008)). A compound that increases levels of T_{H}17-derived cytokines, e.g., IL-17, IL-21 (Spolski and Leonard, Curr. Op. Immunol. 20:295-301 (2008)), or IL-22, is a useful compound.

### Methods of Treatment

As described above, the present invention is based, at least in part, on the identification of certain AHR ligands as compounds that increase levels and/or activity of T_{H}17 cells. Accordingly, the present invention provides compositions and methods for treating a subject (e.g., a human) with a condition that would benefit from an enhanced immune response e.g., a condition caused or associated with an absent or insufficient T_{H}17-mediated immune response. The methods can include selecting a subject in need of treatment (e.g., selecting the subject on the basis that they have one or more conditions that would benefit from an enhanced T_{H}17-mediated immune response) and administering to the subject one or more of the compositions described herein that include as a therapeutic (active) agent an AHR ligand that increases levels or activity of T_{H}17 cells. A subject in need of treatment can be identified, e.g., by their medical practitioner.

Disorders Caused by an Absent or Insufficient T_{H}17-Mediated Immune Response

Absent or insufficient immune responses may be caused, e.g., by a disease that affects the immune system (e.g., HIV and cancer), by evasion of the host immune response by the invading pathogen, or by tolerance to the immune response. Diseases caused by or resulting from an absent or insufficient immune response that may benefit from treatment using the compositions and methods described herein include, but are not limited to, infection (e.g., bacterial (e.g., Klebsiella pneumonia), viral, fungal (e.g., Candida albicans), and protozoal infections). A number of infections known to trigger a T_{H}17-mediated immune response are known in the art, see, e.g., O'Quinn and Palmer, Adv. Immunol., 99:115-163 (2008).

The methods can also be used to treat subjects who are immunodeficient, e.g., subjects who are infected with human immunodeficiency virus (HIV). In subjects infected with HIV, a deficit of T_{H}17 cells is often seen, particularly in those subjects progressing to AIDS (see, e.g., Brenchley et al., Blood, 112:2826-2835 (2008); Douek et al., Annu. Rev. Med. 60:471-84 (2009); Brenchley and Douek, Muc. Immunol. 1(1):23-30 (2008); Cecchinato et al., Muc. Immunol. 1(4):279-288 (2008)). The present methods are particularly useful for those subjects, as well as subjects who are immunodeficient for other reasons, e.g., subjects who are malnourished, are elderly or very young (e.g., infants under 12 months of age) (see, e.g., Siegrist and Aspinall, Nat. Rev. Immunol. 9:185-194 (2009)), or are undergoing chemotherapy that results in immune suppression. Some subjects who are immunodeficient due to a genetic mutation, e.g., autosomal dominant hyper-IgE syndrome (HIES, ‘Job's syndrome’), which is associated with a mutation in STAT3 (see, e.g., Milner et al., Nature 452:773-777 (2008)), can also be treated using a method described herein. For those subjects in whom direct administration of an active compound is insufficient, the methods can include administering a population of T_{H}17 cells obtained in vitro using a method described herein.

In addition, the methods described herein can be used to treat subjects with cancer, e.g., with carcinoma (defined as cancer that begins in the skin or in tissues that line or cover internal organs); sarcoma (defined as cancer that begins in bone, cartilage, fat, muscle, blood vessels, or other connective or supportive tissue); leukemia (defined as cancer that starts in blood-forming tissue such as the bone marrow and causes large numbers of abnormal blood cells to be produced and enter the blood); lymphoma and myeloma (defined as cancers that begin in the cells of the immune system); or central nervous system cancers (defined as cancers that begin in the tissues of the brain and spinal cord).

In some embodiments, the methods described herein can be used to treat subjects suffering from one or more of the following: malignant tumors of the nasal cavity, nasal sinuses, nasopharynx, larynx, trachea, bronchi, lungs, jawbones, skin, ear, bones, thyroid gland, prostate gland, ovaries, the Bartholin gland, vulva, vagina, uterine tubes, uterine body, womb, cervical, breast, urinary bladder, kidneys, gall bladder, rectum, colon, appendix, small intestine, stomach, esophagus, or sialadens.

As described herein, diseases or disorders caused by an absent or insufficient immune response can be treated by increasing the number of T_{H}17 cells and/or the activity of T_{H}17 cells in a subject using a therapeutically effective amount of one or more AHR ligands that reduce expression of Foxp3 (e.g., 6-formylindolo[3,2-b]carbazole (FICZ) or beta-naphthoflavone (bNF), and compounds with the same effect on AHR signaling as FICZ or bNF), that are capable of promoting an increase in the number or activity of T_{H}17 cells in vitro and/or in vivo.

In some embodiments, a subject in need of treatment can be administered a pharmaceutically effective dose of one or more AHR ligands that reduce expression of Foxp3 (e.g., FICZ or bNF) capable of promoting an increase in the number or activity of T_{H}17 cells in vitro and/or in vivo.

Alternatively or in addition, a population of cells capable of differentiation into T_{H}17 cells (e.g., naïve T cells and/or CD4⁺CD62 ligand⁺ T cells) can be contacted in vitro with an AHR ligand (e.g., FICZ or bNF, or a compound with the same effect on AHR signaling as FICZ or bNF), thereby effectively promoting an increase in the number of T_{H}17 cells in the population. Alternatively or in addition, a population of cells containing T_{H}17 cells (e.g., isolated T_{H}17 cells (e.g., 100%) or a population of cells containing at least 20, 30, 40, 50, 60, 70, 80, 90, 95, or 99% T_{H}17 cells) can be contacted with FICZ or bNF or a compound with the same effect on AHR signaling as FICZ or bNF, thereby effectively promoting an increase in the activity of the T_{H}17 cells in the population. When administered in vitro, the AHR ligand (e.g., FICZ or bNF) will generally be co-administered with one or both of IL-6 and TGF-beta. (As these compounds are present in vivo, they need not be, but can optionally, administered when the AHR ligand (e.g., FICZ or bNF) is administered in vivo.) One or more cells from these populations can then be administered to the subject alone or in combination with one or more AHR ligands capable of promoting an increase in the number or activity of T_{H}17 cells in vitro and/or in vivo (e.g., FICZ or bNF, or a compound with the same effect on AHR signaling as FICZ or bNF).

Validation of Treatment/Monitoring Treatment Efficacy

During and/or following treatment, a subject can be assessed at one or more time points, for example, using methods known in the art for assessing severity of the disease or its symptoms, to determine the effectiveness of the treatment. In some embodiments, levels of T cells producing IL-17 (T_{H}17) can be evaluated. Treatment can then be continued without modification, modified to improve the progress or outcome (e.g., increase dosage levels, frequency of administration, the amount of the pharmaceutical composition, and/or change the mode of administration), or stopped.

A number of methods of evaluation of efficacy can be used, e.g., detection of levels of (RORγt), a transcription factor associated with T_{H}17 cell differentiation, e.g., using RT-PCR or intracellular staining/FACS analysis (see, e.g., Ivanov et al., Nat. Immunol., 8:345-50, 2007); alternatively or in addition, levels of IL-17, IL-21, or IL-22 can also be evaluated, e.g., using intracellular cytokine staining, ELISA, ELISPOT, or RT-PCR assays as known in the art. Clinical parameters, e.g., tumor size or growth, infection control or levels of a pathogen present (also known as “load”), can also be evaluated.

Administration

A therapeutically effective amount of one or more of the compositions described herein can be administered by standard methods, for example, by one or more routes of administration, e.g., by one or more of the routes of administration currently approved by the United States Food and Drug Administration (FDA; see, for example world wide web address fda.gov/cder/dsm/DRG/drg00301.htm), e.g., orally, topically, mucosally, intravenously or intramuscularly.

In some embodiments, one or more of the ligands described herein can be administered orally with surprising effectiveness.

### AHR Ligand-Nanoparticles

As demonstrated herein, compositions comprising nanoparticles linked to AHR ligands (e.g. FICZ or bNF) are surprisingly effective in delivering the ligand, both orally and by injection, and in inducing the Treg response in living animals. Thus, the invention further includes compositions comprising AHR ligands linked to biocompatible nanoparticles, optionally with antibodies that target the nanoparticles to selected cells or tissues.

Biocompatible Nanoparticles

The nanoparticles useful in the methods and compositions described herein are made of materials that are (i) biocompatible, i.e., do not cause a significant adverse reaction in a living animal when used in pharmaceutically relevant amounts; (ii) feature functional groups to which the binding moiety can be covalently attached, (iii) exhibit low non-specific binding of interactive moieties to the nanoparticle, and (iv) are stable in solution, i.e., the nanoparticles do not precipitate. The nanoparticles can be monodisperse (a single crystal of a material, e.g., a metal, per nanoparticle) or polydisperse (a plurality of crystals, e.g., 2, 3, or 4, per nanoparticle).

A number of biocompatible nanoparticles are known in the art, e.g., organic or inorganic nanoparticles. Liposomes, dendrimers, carbon nanomaterials and polymeric micelles are examples of organic nanoparticles. Quantum dots can also be used. Inorganic nanoparticles include metallic nanoparticle, e.g., Au, Ni, Pt and TiO2 nanoparticles. Magnetic nanoparticles can also be used, e.g., spherical nanocrystals of 10-20 nm with a Fe²⁺ and/or Fe³⁺ core surrounded by dextran or PEG molecules. In some embodiments, colloidal gold nanoparticles are used, e.g., as described in Qian et al., Nat. Biotechnol. 26(1):83-90 (2008); U.S. Pat. Nos. 7,060,121; 7,232,474; and U.S. P.G. Pub. No. 2008/0166706. Suitable nanoparticles, and methods for constructing and using multifunctional nanoparticles, are discussed in e.g., Sanvicens and Marco, Trends Biotech., 26(8): 425-433 (2008).

In all embodiments, the nanoparticles are attached (linked) to the AHR ligands described herein via a functional groups. In some embodiments, the nanoparticles are associated with a polymer that includes the functional groups, and also serves to keep the metal oxides dispersed from each other. The polymer can be a synthetic polymer, such as, but not limited to, polyethylene glycol or silane, natural polymers, or derivatives of either synthetic or natural polymers or a combination of these. Useful polymers are hydrophilic. In some embodiments, the polymer “coating” is not a continuous film around the magnetic metal oxide, but is a “mesh” or “cloud” of extended polymer chains attached to and surrounding the metal oxide. The polymer can comprise polysaccharides and derivatives, including dextran, pullanan, carboxydextran, carboxmethyl dextran, and/or reduced carboxymethyl dextran. The metal oxide can be a collection of one or more crystals that contact each other, or that are individually entrapped or surrounded by the polymer.

In other embodiments, the nanoparticles are associated with non-polymeric functional group compositions. Methods are known to synthesize stabilized, functionalized nanoparticles without associated polymers, which are also within the scope of this invention. Such methods are described, for example, in Halbreich et al., Biochimie, 80 (5-6):379-90, 1998.

In some embodiments, the nanoparticles have an overall size of less than about 1-100 nm, e.g., about 25-75 nm, e.g., about 40-60 nm, or about 50-60 nm in diameter. The polymer component in some embodiments can be in the form of a coating, e.g., about 5 to 20 nm thick or more. The overall size of the nanoparticles is about 15 to 200 nm, e.g., about 20 to 100 nm, about 40 to 60 nm; or about 60 nm.

Synthesis of Nanoparticles

There are varieties of ways that the nanoparticles can be prepared, but in all methods, the result must be a nanoparticle with functional groups that can be used to link the nanoparticle to the binding moiety.

For example, AHR ligands can be linked to the metal oxide through covalent attachment to a functionalized polymer or to non-polymeric surface-functionalized metal oxides. In the latter method, the nanoparticles can be synthesized according to a version of the method of Albrecht et al., Biochimie, 80(5-6): 379-90, 1998. Dimercapto-succinic acid is coupled to the nanoparticle and provides a carboxyl functional group. By functionalized is meant the presence of amino or carboxyl or other reactive groups that can be used to attach desired moieties to the nanoparticles, e.g., the AHR ligands described herein or antibodies.

In another embodiment, the AHR ligands are attached to the nanoparticles via a functionalized polymer associated with the nanoparticle. In some embodiments, the polymer is hydrophilic. In a specific embodiment, the conjugates are made using oligonucleotides that have terminal amino, sulfhydryl, or phosphate groups, and superparamagnetic iron oxide nanoparticles bearing amino or carboxy groups on a hydrophilic polymer. There are several methods for synthesizing carboxy and amino derivatized-nanoparticles. Methods for synthesizing functionalized, coated nanoparticles are discussed in further detail below.

Carboxy functionalized nanoparticles can be made, for example, according to the method of Gorman (see WO 00/61191). Carboxy-functionalized nanoparticles can also be made from polysaccharide coated nanoparticles by reaction with bromo or chloroacetic acid in strong base to attach carboxyl groups. In addition, carboxy-functionalized particles can be made from amino-functionalized nanoparticles by converting amino to carboxy groups by the use of reagents such as succinic anhydride or maleic anhydride.

Nanoparticle size can be controlled by adjusting reaction conditions, for example, by varying temperature as described in U.S. Pat. No. 5,262,176. Uniform particle size materials can also be made by fractionating the particles using centrifugation, ultrafiltration, or gel filtration, as described, for example in U.S. Pat. No. 5,492,814.

Nanoparticles can also be treated with periodate to form aldehyde groups. The aldehyde-containing nanoparticles can then be reacted with a diamine (e.g., ethylene diamine or hexanediamine), which will form a Schiff base, followed by reduction with sodium borohydride or sodium cyanoborohydride.

Dextran-coated nanoparticles can also be made and cross-linked, e.g., with epichlorohydrin. The addition of ammonia will react with epoxy groups to generate amine groups, see Hogemann et al., Bioconjug. Chem. 2000. 11(6):941-6, and Josephson et al., Bioconjug. Chem., 1999, 10(2):186-91.

Carboxy-functionalized nanoparticles can be converted to amino-functionalized magnetic particles by the use of water-soluble carbodiimides and diamines such as ethylene diamine or hexane diamine.

Avidin or streptavidin can be attached to nanoparticles for use with a biotinylated binding moiety, such as an oligonucleotide or polypeptide. See e.g., Shen et al., Bioconjug. Chem., 1996, 7(3):311-6. Similarly, biotin can be attached to a nanoparticle for use with an avidin-labeled binding moiety.

In all of these methods, low molecular weight compounds can be separated from the nanoparticles by ultra-filtration, dialysis, magnetic separation, or other means. The unreacted AHR ligands can be separated from the ligand-nanoparticle conjugates, e.g., by size exclusion chromatography.

In some embodiments, colloidal gold nanoparticles are made using methods known in the art, e.g., as described in Qian et al., Nat. Biotechnol. 26(1):83-90 (2008); U.S. Pat. Nos. 7,060,121; 7,232,474; and U.S. P.G. Pub. No. 2008/0166706.

In some embodiments, the nanoparticles are pegylated, e.g., as described in U.S. Pat. Nos. 7,291,598; 5,145,684; 6,270,806; 7,348,030, and others.

### Antibodies

In some embodiments, the compositions described herein also include antibodies to selectively target a cell; in some embodiments, the antibodies are bound to nanoparticles, e.g., the same or different nanoparticles as the AHR ligand. The term “antibody,” as used herein, refers to full-length, two-chain immunoglobulin molecules and antigen-binding portions and fragments thereof, including synthetic variants. A typical full-length antibody includes two heavy (H) chain variable regions (abbreviated herein as VH), and two light (L) chain variable regions (abbreviated herein as VL). The term “antigen-binding fragment” of an antibody, as used herein, refers to one or more fragments of a full-length antibody that retain the ability to specifically bind to a target. Examples of antigen-binding fragments include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546 (1989)), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science 242:423-426 (1988); and Huston et al. Proc. Natl. Acad. Sci. USA 85:5879-5883 (1988)). Such single chain antibodies are also encompassed within the term “antigen-binding fragment.”

Production of antibodies and antibody fragments is well documented in the field. See, e.g., Harlow and Lane, 1988. Antibodies, A Laboratory Manual. Cold Spring Harbor, N.Y.: Cold Spring Harbor Laboratory. For example, Jones et al., Nature 321: 522-525 (1986), which discloses replacing the CDRs of a human antibody with those from a mouse antibody. Marx, Science 229:455-456 (1985), discusses chimeric antibodies having mouse variable regions and human constant regions. Rodwell, Nature 342:99-100 (1989), discusses lower molecular weight recognition elements derived from antibody CDR information. Clackson, Br. J. Rheumatol. 3052: 36-39 (1991), discusses genetically engineered monoclonal antibodies, including Fv fragment derivatives, single chain antibodies, fusion proteins chimeric antibodies and humanized rodent antibodies. Reichman et al., Nature 332: 323-327 (1988) discloses a human antibody on which rat hypervariable regions have been grafted. Verhoeyen, et al., Science 239: 1534-1536 (1988), teaches grafting of a mouse antigen binding site onto a human antibody.

In the methods described herein, it would be desirable to target the compounds to T cells, B cells, dendritic cells, and/or macrophages, therefore antibodies selective for one or more of those cell types can be used. For example, for T cells, anti-CXCR4, anti-CD28, anti-CD8, anti-TTLA4, or anti-CD3 antibodies can be used; for B cells, antibodies to CD20, CD19, or to B-cell receptors can be used; for dendritic cell targeting, exemplary antibodies to CD11c, DEC205, MHC class I or class II, CD80, or CD86 can be used; for macrophages, exemplary antiboduies to CD11b, MHC class I or class 11, CD80, or CD86 can be used. Other suitable antibodies are known in the art.

### Pathogen- and Tumor-Specific Antigens

In some embodiments, e.g., where a population of cells is administered to a subject, the methods include co-administering a specific antigen, to induce an antigen-specific response. Thus, for example, where the subject has a tumor, one or more tumor-specific antigens can be administered, e.g., antigens associated with the type of tumor the subject has.

The specific antigens can be purified, e.g., isolated and purified polypeptides or glycopeptides, e.g., native or recombinant, and can include antigenic fragments as well. Where the subject has a tumor or an infection other than viral, and the antigen is from a tumor cell, bacteria, fungus, or protozoa, i.e., a cell-associated antigen, whole cells or fragments thereof can also be administered.

Methods for selecting and preparing specific antigens are well known in the art. For example, any antigen that has been identified as potentially useful as a vaccine can be used. In this case, the methods can include administering the AHR ligand, or T_{H}17 cells prepared by a method described herein, as part of a vaccination protocol, e.g., as an adjuvant to boost the immune response to the vaccine antigen. Thus the present methods can be incorporated into any known vaccination protocol, for administration as an adjuvant.

Exemplary tumor-associated antigens (TAAs) useful in the present compositions and methods include those that can be classified as one of the following:
1. Products of Mutated Oncogenes and Tumor Suppressor Genes;
2. Products of Other Mutated Genes;
3. Overexpressed or Aberrantly Expressed Cellular Proteins;
4. Tumor Antigens Produced by Oncogenic Viruses;
5. Oncofetal Antigens;
6. Altered Cell Surface Glycolipids and Glycoproteins; or
7. Cell Type-Specific Differentiation Antigens

Examples of TAAs include the following: alphafetoprotein (AFP), for germ cell tumors; carcinoembryonic antigen (CEA), for cancers of the gastrointestinal tract; CA-125, for ovarian cancer; MUC-1, for breast cancer; epithelial tumor antigen (ETA), for breast cancer; tyrosinase, for malignant melanoma; melanoma-associated antigen (MAGE), for malignant melanoma; prostatic acid phosphatase or prostate specific antigen (PSA), for prostate cancer; or Melan-A/MART-1, for malignant melanoma. Others include abnormal products of ras, or p53; hormones, e.g., ACTH, calcitonin, and human chorionic gonadotropin (HCG); Tumor associated glycoproteins CA 125, CA 19-9, CA 72-4, and CA 15-3.

Exemplary pathogen-associated antigens include antigenic polysaccharides which could be given (conjugated to protein carrier) together with FICZ, to protect children and elders against the causative agents of diseases, e.g., meningitis, e.g., linked to a peptide carrier, see, e.g., Amir-Kroll et al., J. Immunol. 170:6165-6171 (2003). Exemplary polysaccharides include the surface polysaccharides Streptococcal pneumoniae; Neisseria meningitides; and Haemophilus Influenza Type b (Hib).

Other exemplary antigens include Bordatella pertussis formalin-inactivated pertussis toxins, e.g., after removal of cells from culture (acellular pertussis, aP); Clostridium tetani formalin-inactivated toxin; Corynebacterium diphtheriae formalin-inactivated toxins; Hepatitis B virus antigen (HBsAg); and various inactivated viruses/bacteria. A number of other antigens are known in the art.

### Pharmaceutical Formulations

A therapeutically effective amount of one or more of the compositions described herein (i.e., that include as an active (therapeutic) agent an AHR ligand, e.g., FICZ or bNF, either alone or bound to a nanoparticle) can be incorporated into pharmaceutical compositions suitable for administration to a subject, e.g., a human. Such compositions typically include the composition and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions, e.g., an inhibitor of degradation of the ligand.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (polyethoxylated castor oil; BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the composition (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGELT™ (sodium carboxymethyl starch), or corn starch; a lubricant such as magnesium stearate or STEROTES™; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Nucleic acid molecules can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., PNAS 91:3054-3057, 1994). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration. In one aspect, the pharmaceutical compositions can be included as a part of a kit.

Generally the dosage used to administer a pharmaceutical compositions facilitates an intended purpose for prophylaxis and/or treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., Chapter 27 In: “Remington's Pharmaceutical Sciences”, 18th Ed., Gennaro, ed., Mack Publishing Co., Easton, Pa., 1990). Generally, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3, In: Goodman & Gilman's “The Pharmacological Basis of Therapeutics”, 9th Ed., Hardman et al., eds., McGraw-Hill, New York, N.Y., 1996).

### Kits

The present invention also includes kits. In some embodiments the kit comprise one or more doses of a composition described herein. The composition, shape, and type of dosage form for the induction regimen and maintenance regimen may vary depending on a subjects requirements. For example, dosage form may be a parenteral dosage form, an oral dosage form, a delayed or controlled release dosage form, a topical, and a mucosal dosage form, including any combination thereof.

In a particular embodiment, a kit can contain one or more of the following in a package or container: (1) one or more doses of a composition described herein; (2) one or more pharmaceutically acceptable adjuvants or excipients (e.g., a pharmaceutically acceptable salt, solvate, hydrate, stereoisomer, and clathrate); (3) one or more vehicles for administration of the dose; (5) instructions for administration. Embodiments in which two or more, including all, of the components (1)-(5), are found in the same container can also be used.

When a kit is supplied, the different components of the compositions included can be packaged in separate containers and admixed immediately before use. Such packaging of the components separately can permit long term storage without loosing the active components' functions. When more than one bioactive agent is included in a particular kit, the bioactive agents may be (1) packaged separately and admixed separately with appropriate (similar of different, but compatible) adjuvants or excipients immediately before use, (2) packaged together and admixed together immediately before use, or (3) packaged separately and admixed together immediately before use. If the chosen compounds will remain stable after admixing, the compounds may be admixed at a time before use other than immediately before use, including, for example, minutes, hours, days, months, years, and at the time of manufacture.

The compositions included in particular kits of the present invention can be supplied in containers of any sort such that the life of the different components are optimally preserved and are not adsorbed or altered by the materials of the container. Suitable materials for these containers may include, for example, glass, organic polymers (e.g., polycarbonate and polystyrene), ceramic, metal (e.g., aluminum), an alloy, or any other material typically employed to hold similar reagents. Exemplary containers may include, without limitation, test tubes, vials, flasks, bottles, syringes, and the like.

As stated above, the kits can also be supplied with instructional materials. These instructions may be printed and/or may be supplied, without limitation, as an electronic-readable medium, such as a floppy disc, a CD-ROM, a DVD, a Zip disc, a video cassette, an audiotape, and a flash memory device. Alternatively, instructions may be published on a internet web site or may be distributed to the user as an electronic mail.

### EXAMPLES

The invention is further described in the following examples, which do not limit the scope of the invention described in the claims.

### Example 1

### Role of AHR on T_{H}17 Differentiation In Vitro

It has been recently reported that UVB light exposed keratinocytes up-regulate surface RANKL levels, signaling epidermal dendritic cells to support Treg expansion (Loser et al., Nat. Med., 12:1372-1379, 2006). UVB catalyzes the formation of the AHR ligand FICZ in vitro, and is thought to generate FICZ in the skin (Fritsche et al., Proc. Natl. Acad. Sci. U.S.A., 104:8851-8856, 2007). In light of the role of AHR on Treg (see PCT International Patent Application NO. PCT/US2008/083016, and U.S. Provisional Patent Application Ser. No. 60/989,309, filed on Nov. 20, 2007, both of which are incorporated herein by reference in their entirety), and the reported effects of UVB on Treg expansion (Loser et al., Nat. Med., 12:1372-1379, 2006), the effects of FICZ on Treg were investigated.

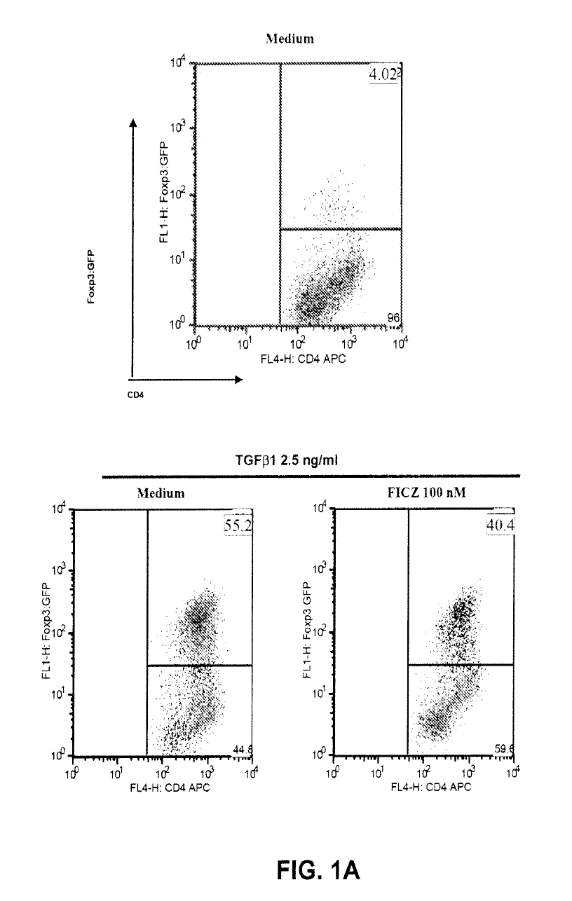

As shown in FIGS. 1A and 1B, surprisingly, rather than promoting the conversion of CD4⁺ Foxp3:GFP⁻ T cells into CD4⁺ Foxp3:GFP⁻ Treg, FICZ instead interfered with the differentiation of Treg triggered by TGFβ1 in vitro. Based on the reported reciprocal relationship that exists between T_{H}17 and Treg cells (Bettelli et al., Nature, 441:235-238, 2006), and the inhibitory effects of FICZ on Treg differentiation described herein, the role of FICZ and AHR on T_{H}17 differentiation was investigated.

As shown in FIG. 1C, AHR expression is highly up-regulated in T_{H}17 T cells induced in vitro by activation with TGFβ1 and IL-6. Moreover, AHR expression was also up-regulated when T_{H}17 differentiation was driven by TGFβ1, IL-6 and IL-23, or when IL-21 was used instead of IL-6 (see FIG. 1D).

Next, the effect of AHR activation by FICZ on in vitro T_{H}17 differentiation was investigated using the T_{H}17 transcription factor RORyt as a marker of T_{H}17 differentiation. As shown in FIG. 1E, FICZ alone did not significantly upregulate the expression levels of the T_{H}17 transcription factor RORyt. However, as shown in FIGS. 1F-1H, FICZ synergized with TGFβ1, IL-6 and IL-23 to drive T_{H}17 differentiation in vitro. This synergism could be blocked with the AHR antagonist resveratrol, as shown in FIG. 1I. This observation demonstrates that the AHR is critical for FICZ-mediated T_{H}17 differentiation. Taken together, these results demonstrate that FICZ promotes T_{H}17 differentiation in vitro.

### Example 2

### Role of AHR on T_{H}17 Differentiation In Vivo

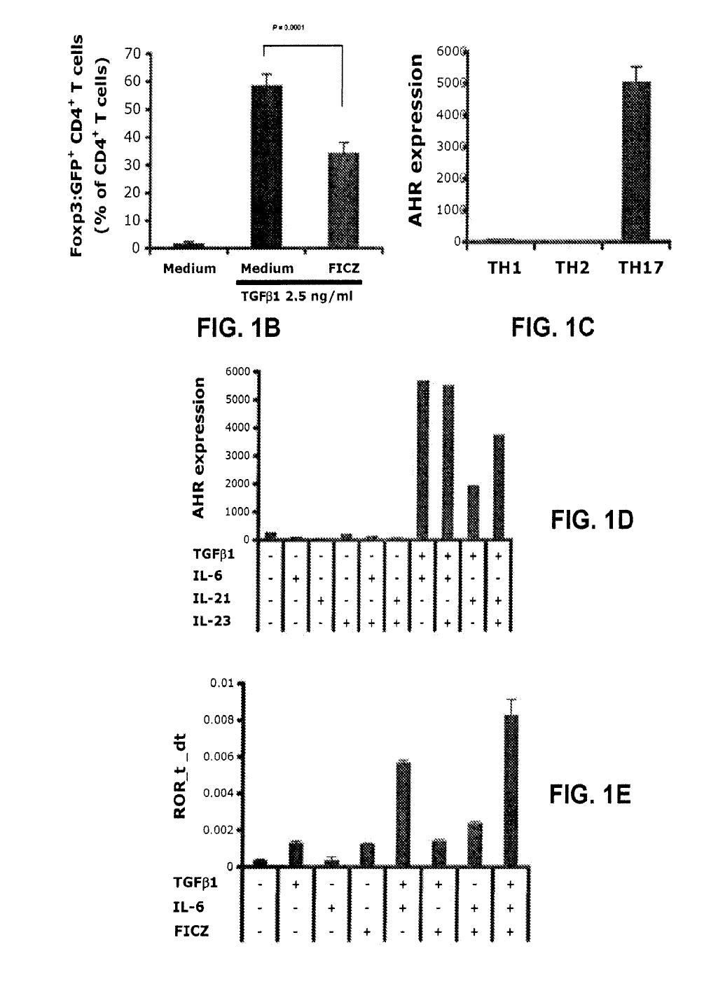

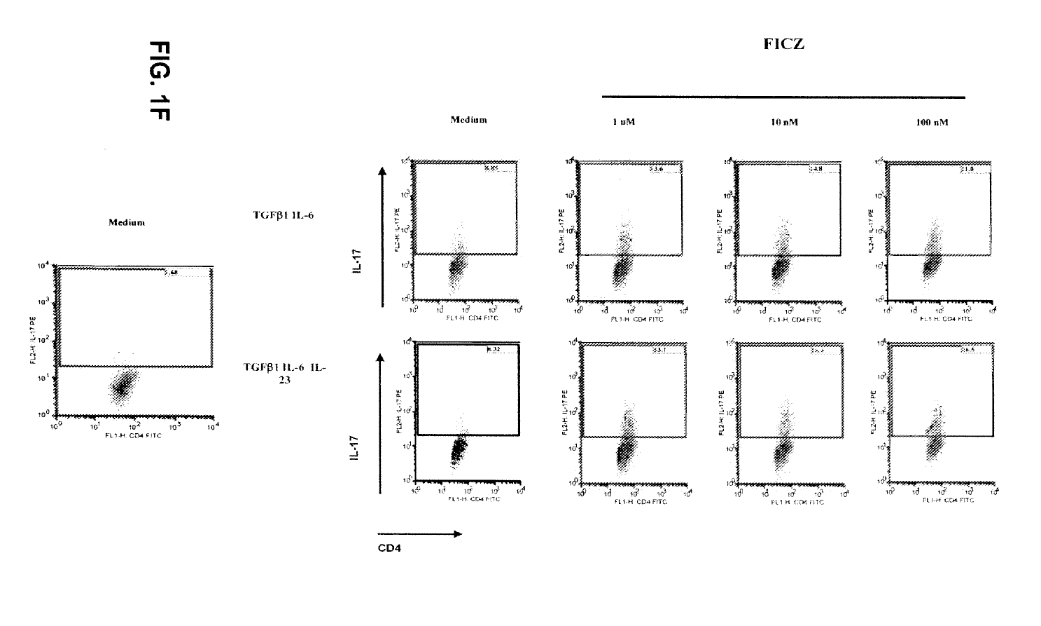

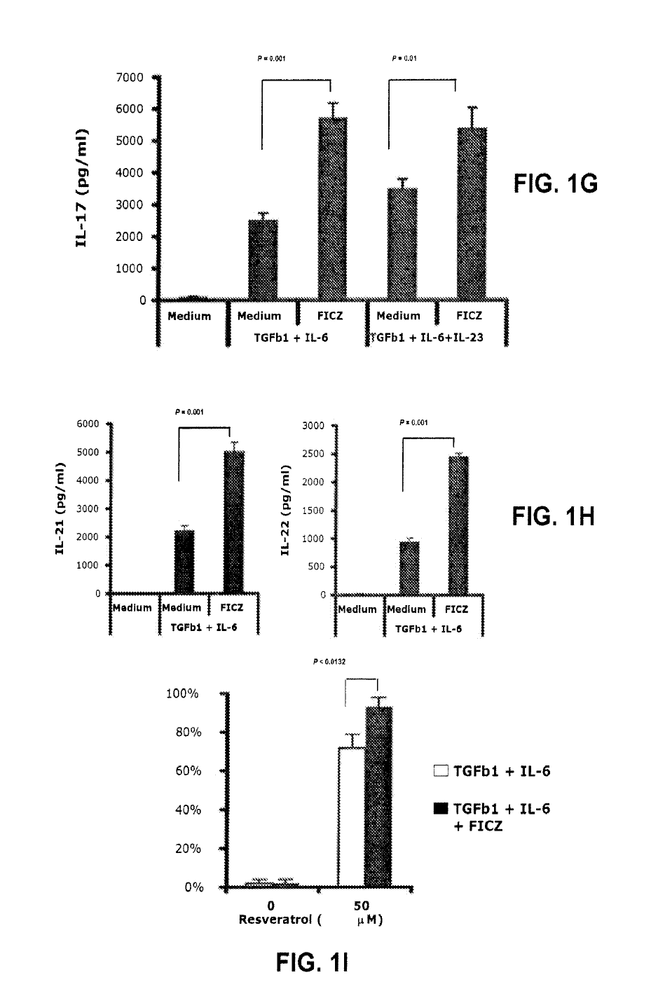

The effects of FICZ were evaluated in vivo using the EAE model described in PCT International Patent Application NO. PCT/US2008/083016, and U.S. Provisional Patent Application Ser. No. 60/989,309, filed on Nov. 20, 2007. As shown in FIG. 2A and Table 1, FICZ administration resulted in a significant worsening of EAE. As shown in FIGS. 2B-2D, FICZ administration was also associated with increased frequencies of IL-17⁺ CD4⁺ and CD4⁺IFNγ⁺ T cells, and increased secretion of IL-17 and IFNγ following in vitro stimulation with MOG₃₅₋₅₅ (see FIG. 2B). Consistent with the reciprocal relationship that exists between T_{H}17 and Treg, FICZ-treated mice showed a decrease in the frequency of CD4⁺Foxp3⁺ Treg (FIG. 2E). Taken together, these results demonstrate that FICZ promotes T_{H}17 differentiation in vivo.

TABLE 1
Effect of FICZ Treatment on EAE

| | Incidence | Mean day of onset | Mean maximum |
|---|---|---|---|
| Treatment | (positive/total) | (Mean ± SD) | score (mean ± SD) |
| Control | 11/14 (79%) | 13.7 ± 1.9 | 2 ± 1.4 |
| FICZ | 12/15 (80%) | 13.3 ± 1.5 | 2.7 ± 1.8 |

Mice were treated with PBS (control) or ITE, immunized with MOG₃₅₋₅₅ peptide in CFA and monitored for EAE development.

### Example 3

### The Effect of FICZ on an in Vivo Model of Hepatocellular Carcinoma

In this example the effects of FICZ administration on Hepatocellular Carcinoma were evaluated in a mouse model of cancer.

The present study used athymic nude mouse (N=7), a type of laboratory mouse that is hairless, lacks a normal thymus gland, and has a defective immune system because of a genetic mutation. Athymic nude mice are often used in cancer research because they do not reject tumor cells, from mice or other species.

100 ug of frozen FICZ (BIOMOL International, Plymouth Meeting, Pa.) was dissolved in 5 ml of CREMOPHOR™ (polyethoxylated castor oil):ethanol in phosphate buffered saline (PBS), to produce a working solution of 20 ug/ml. 250 ul (5 ug total) of HBs antigen was injected in Treatment Group C. The experiments were carried out as follows:

| | |
|---|---|
| Day 1 | Transplantation of HCC (subcutaneously into the back), |
| | 5 × 10⁶ Hep3b cells per mouse) |
| Day 7 | Transplantation (i.v) of whole splenocytes - 1 × 10⁶ |
| | cells per mouse to reconstitute immune system |
| Day 15 | Treatment: twice a week (until week 4)- depending on tumor |
| Day 21 | progression |
| Day 28 | Group A: Control (CREMOPHOR ™ (polyethoxylated castor |
| Day 35 | oil, BASF Corp.) vehicle only, CREMOPHOR: ethanol, IP, |
| | 50 ul) |
| | Group B: FICZ, IP (50 ul) |
| | Group C: HBsAg (hepatitis B surface antigen) |
| | (Energix) + FICZ − IP (250 ul + 50 ul) |
| WEEKS | Every week for a total of 4 weeks, measured: |
| 2-5 | Survival |
| | Weight |
| | Tumor volume (3 dimensions) |
| | Alpha Fetoprotein (AFP) serum levels |
| | Tumor histology for inflammation and apoptosis |
| | Keep frozen and paraffin sections for in situ from |
| | any animals that |

Tumor sizes were measured in three dimensions using Vernier calipers and tumor volume was computed assuming a spherical geometry with radius equal to one-half the average tumor dimension: TV (mm³)=d²×D/2. Tumors were measured two times a week and mice were monitored routinely to evaluate the effects of treatment. The results demonstrated that the administration of FICZ suppressed tumor growth.

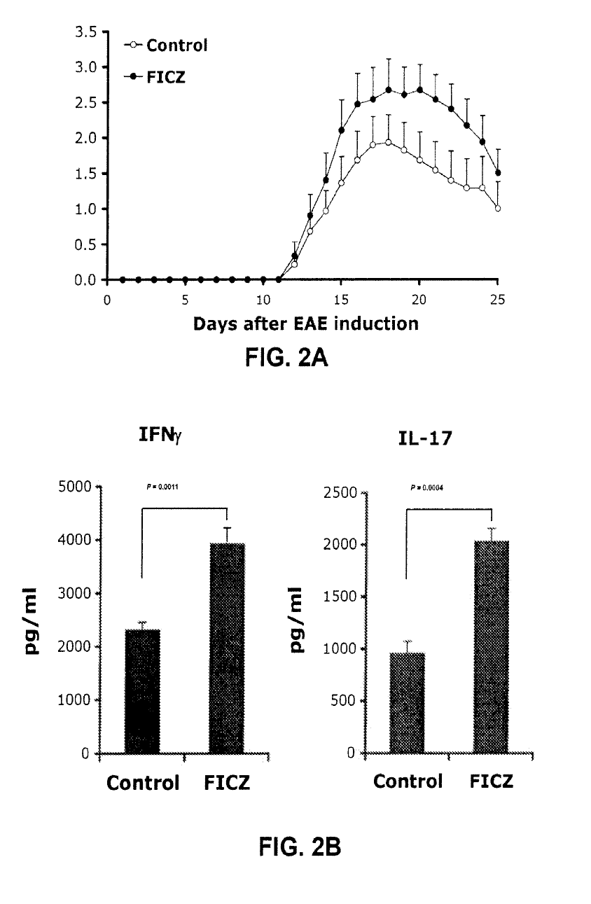

In addition, serum AFP levels were monitored; AFP is an accepted tumor marker of HCC. The results are shown in FIGS. 3A and 3B. As measured by AFP levels at seven (FIG. 3A) and 14 (FIG. 3B) days after treatment initiation, FICZ significantly suppresses tumor growth. In addition, a combination treatment comprising administering FICZ with HBsAG provided somewhat better results than FICZ alone, which indicates that the co-administration of a tumor associated antigen has a synergistic effect.

At week 6, the animals are sacrificed, and FACS sorting is performed on cells obtained from spleen to isolate and purify T cells, and the following are measured: Proliferation of splenocytes against Hep3B and HCC lysate; Cytokines: IFN, IL17, TGF IL6, IL2, IL10; Cytotoxicity of T cells against HBsAg; and perforin in CD4 and CD8.

### Example 4

### T_{H} 17 in the Teleost Danio rerio

The immune system in teleosts like the zebrafish (Danio rerio) resembles in several aspects the mammalian immune system. Macrophages, T cells, B cells have been described in teleosts (Langenau and Zon, Nat Rev Immunol. 5, 307-317 (2005)), as well as the cytokines IL-17 (Gunimaladevi et al., Fish Shellfish Immunol 21, 393-403 (2006)), IFNg (Robertsen, Fish Shellfish Immunol 20, 172-191 (2006)) and TNFa (Clay et al., Immunity 29, 283-294 (2008)) and the transcription factors T-bet (Takizawa et al., Mol Immunol 45, 127-136 (2008)) and retinoid-related orphan receptor (Flores et al., Gene Expr Patterns 7, 535-543 (2007)) which have been linked to mammalian autoimmune pathology. Like in mammals, the immune repertoire of teleosts is generated by recombinatorial and mutational mechanisms (Boehm, Cell 125, 845-858 (2006); Boehm and Bleul, Immunol 8, 131-135 (2007); Cooper and Alder, Cell 124, 815-822 (2006); Langenau and Zon, Nat Rev Immunol. 5, 307-317 (2005); Pancer and Cooper, Annu Rev Immunol. 24, 497-518 (2006)). However, although these processes can generate potentially harmful self-reactive immune receptors, the potential for adaptive autoimmunity and mechanisms of immunoregulation have not yet been characterized in lower gnathostomes. Indeed, Foxp3-driven peripheral tolerance has been postulated to be a recent adaptation in vertebrate evolution (Boehm, Cell 125, 845-858 (2006)).

The autoimmune response in these animals was studied in 6-month old zebrafish immunized intraperitoneally (ip) with zebrafish brain homogenate (zCNS) emulsified in complete Freund's adjuvant (CFA).

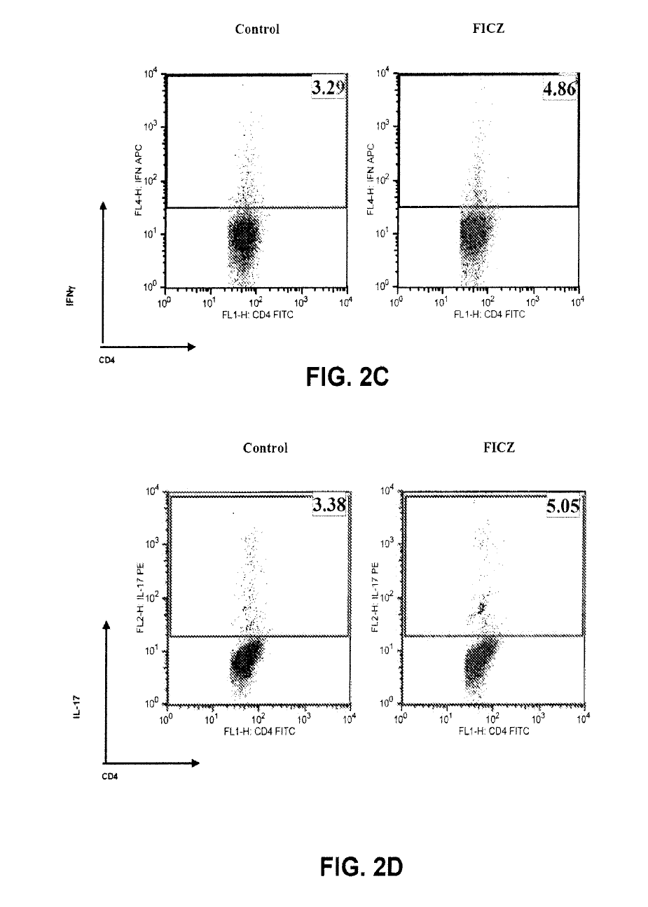

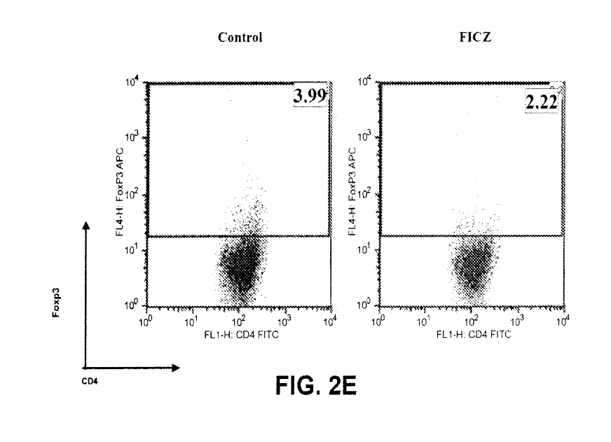

Autoimmune encephalomyelitis was detected as evidenced by the presence of zCNS immunization induced autoantibodies directed against zCNS and its derived peptides (detected using zebrafish myelin microarrays (Quintana et al., Proc Natl Acad Sci USA 101 Suppl 2, 14615-14621 (2004); Robinson et al., Nat Biotechnol. 21, 1033-1039 (2003))), and the accumulation of CD3, IFNgamma and IL-17 expressing cells in the brain of zCNS-immunized zebrafish (FIGS. 4A-4C).

These results demonstrate that zebrafish can mount adaptive antigen-specific autoimmune responses.

To further investigate the function of zFoxp3 in zebrafish, zFoxp3 was over-expressed or alternatively knocked out in zebrafish developing embryos.

zFoxp3 was cloned from cDNA prepared from zebrafish kidney by using a TOPO® PCR cloning kit (Invitrogen, CA, USA) according to the manufacturer's instructions.

Zebrafish eggs were collected within 1 hr of spawning, and purified plasmids or morpholino antisense oligonucleotides were microinjected with a fine glass needle connected to an automatic injector. A morpholino oligonucleotide designed to block the translation of zFoxp3 (5′-GTGTTCCAGTAGCATTAAGAAGCAT-3′) and a 5 bases mismatch control oligonucleotide (5′-GTcTTCgAGTAcCATTAAcAAGgAT-3′) were designed and synthesized by Gene Tools (Philomath, Oreg.). Each morpholino nucleotide was injected into the yolk of embryos at one to four cell stages.

Microinjection with zFoxp3-expression constructs resulted in an up-regulation of zFoxp3 levels, concomitant with the down-regulation of IL-17 levels. Conversely, microinjection with morpholino oligonucleotides designed to block the translation of zFoxp3 led to the upregulation of IL-17 expression, which was not observed upon the injection of 5 bases mismatch negative control morpholino (FIG. 4D).

Furthermore, as noted in mammalian cells, the treatment of developing zebrafish embryos with the high-affinity AHR ligand 2,3,7,8-tetrachlorodibenzo-p-dioxin (TCDD) resulted in a dose-dependent increase in zFoxp3 expression; this increase was concomitant with a down regulation in IL-17 expression.

All in all, these results indicate that zFoxp3 is the functional zebrafish homologue of Foxp3 in mammals. Increases in zFoxp3 result in an increase in Treg, while a decrease in Foxp3 expression results in an increase in levels of IL-17.

### Example 4

### Administration of FICZ-Loaded Nanoparticles Worsens EAE

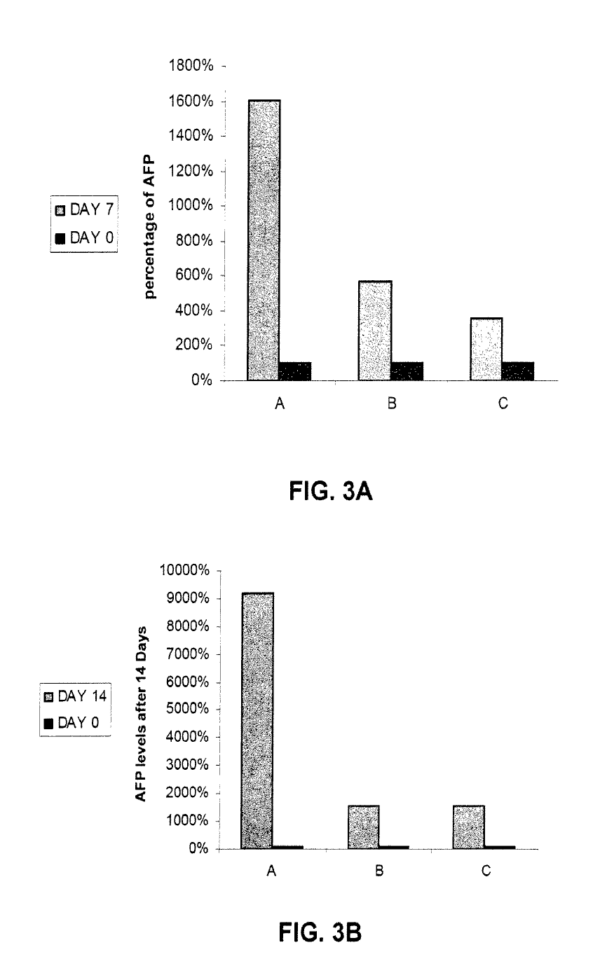

As noted above, administration of a single dose of 1 μg/mouse of the AHR ligand FICZ worsens EAE. Gold colloid has been in use for over 50 years in the treatment of rheumatoid arthritis, these gold colloid nanoparticles have been shown to have little to no long-term toxicity or adverse effects (Paciotti et al., Drug Deliv. 11, 169 (May-June, 2004)). Due to their small size (10-100 nm diameter), gold colloid nanoparticles have large surface areas on which multiple small proteins or other molecules can be conjugated (Paciotti et al., Drug Deliv. 11, 169 (May-June, 2004)). The PEGylation of gold colloid nanoparticles greatly enhances the overall stability of the molecule to which it is covalently bonded (Qian et al., Nat Biotechnol. 26, 83 (January, 2008)). Moreover, recently it has been shown that PEGylated) gold colloid nanoparticles can be linked to specific antibodies to target them to specific cell types (Qian et al., Nat Biotechnol. 26, 83 (January, 2008)). Thus, to increase the half-life of FICZ and to facilitate its targeting to specific cell types, polyethylene glycol coated (PEGylated) gold colloid nanoparticles loaded with AHR ligands were constructed (FIG. 5).

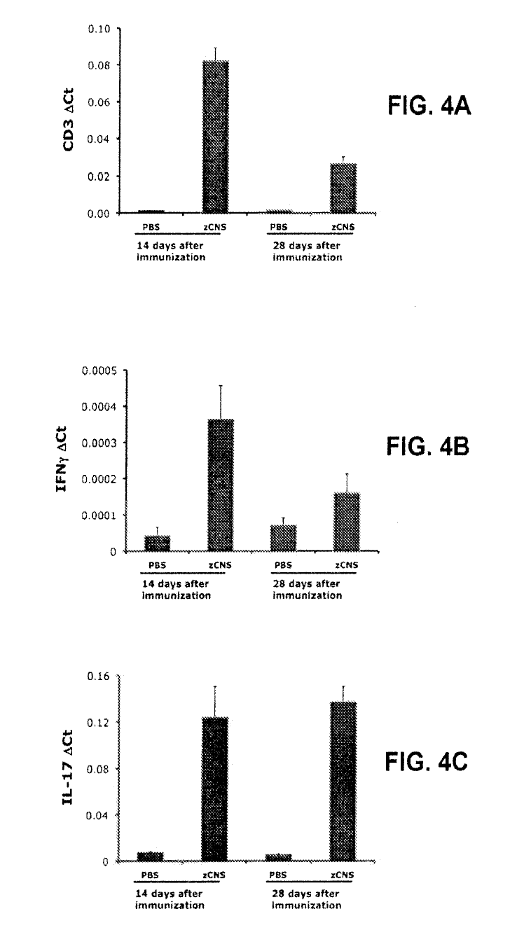

PEGylated gold colloid nanoparticles carrying the AHR ligands FICZ, ITE or TCDD showed a typical spectrum of optical absorption (FIGS. 6A-B). Moreover, FICZ, ITE or TCDD-loaded nanoparticles activated luciferase expression on an AHR-reporter cell line to levels similar to those achieved by 10 nM TCDD.

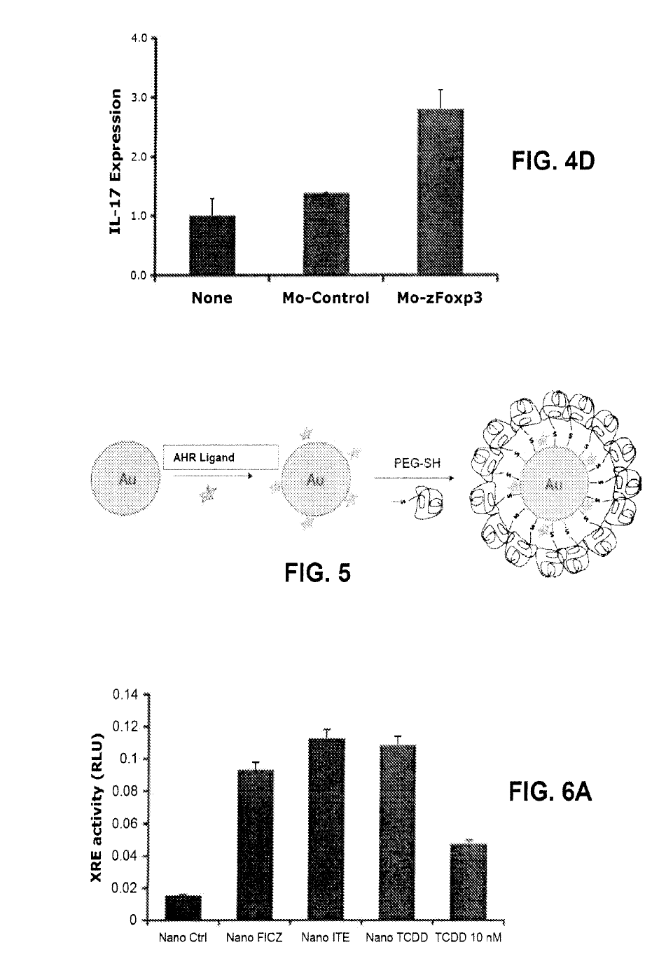

To investigate the in vivo functionality of AHR-ligand loaded nanoparticles EAE was induced on naïve C57BL/6 mice and treated them, starting at day 0, weekly with 45 femtomoles of nanoparticles. Similarly to what was described above, treatment with TCDD resulted in a complete suppression of EAE, while the AHR ligand FICZ worsened the disease (FIG. 7). Weekly administration of ITE-loaded nanoparticles resulted in a significant inhibition of EAE development (FIG. 7).

### Example 5

### An AHR Reporter System for Identification of Modulators of AHR

A construct coding for Foxp3 fused to Renilla luciferase (Ren) was created to provide a simple assay to identify compounds that increase expression of AHR (and thus increase luciferase expression and fluorescence as compared to a control) or that decrease expression of AHR (and thus decrease luciferase expression and fluorescence as compared to a control). HEK 293 cells were transfected as described (Bettelli et al., Proc Natl Acad Sci USA 102, 5138-5143 (2005)) with the AHR reporter luciferase construct and a TK-Renilla Luciferase construct for normalization purposes.

The cells were incubated with different concentrations of the AHR ligand

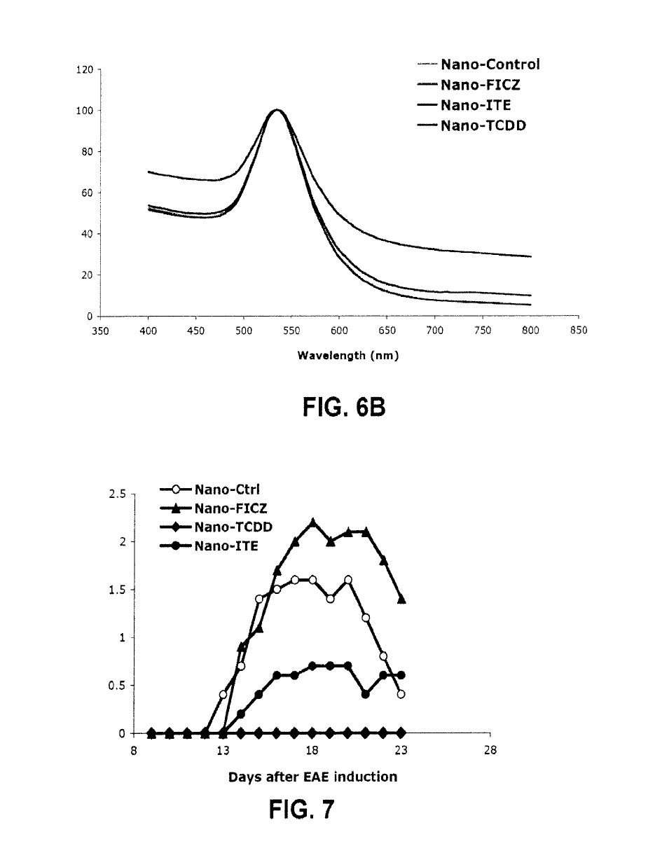

TCDD and activation of the AHR reporter was assayed using the dual luciferase assay kit (New England Biolabs, Ipswich, Mass.). Tk-Renilla was used for standardization. The results, shown in FIG. 8, demonstrate that the construct responds as expected to the AHR ligand TCDD, with dose-dependent increases in expression of the reporter.

### Example 6

### Differentiation of T Cells into T_{H}17 Cells by bNF

To determine whether there are other AHR ligands that would have the same effect on T cell differentiation into T_{H}17 cells, the AHR ligand beta-naphthoflavone (bNF, Sigma-Aldrich) was evaluated.

Briefly, T cells were differentiated into Th17 cells by in vitro activation with antibodies to CD3 and CD28 in the presence of TGF-beta and IL-21 as described (Nature 2008; 454(7202):350-2), in the presence or not of bNF or FICZ (100 nM). IL-17 production was measured by real time PCR.

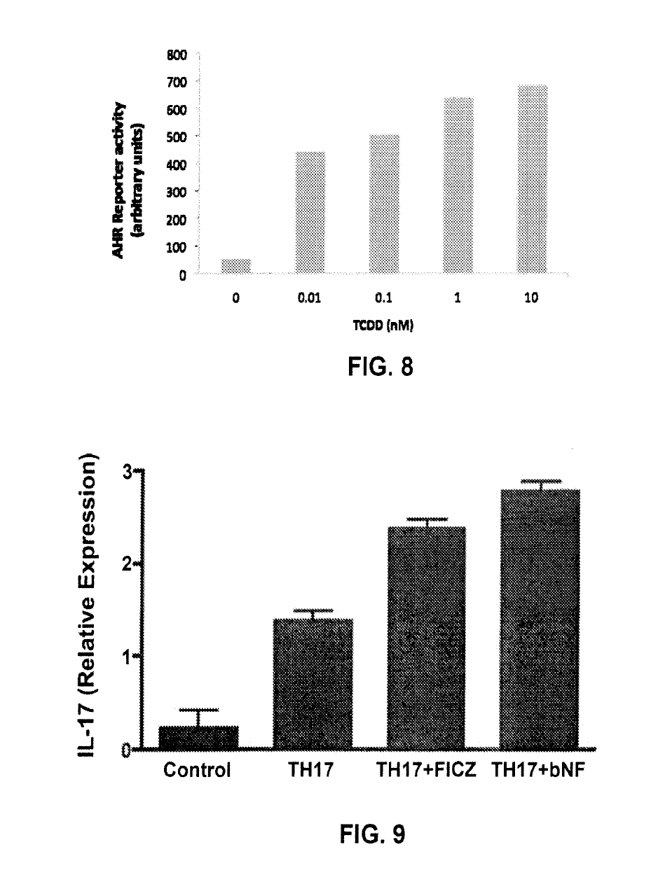

The results, shown in FIG. 9, demonstrate that bNF has the same effect on T cell differentiation into T_{H}17 cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for preparing an enriched population of T cells producing IL-17 (T_{H}17) from an initial population of T cells, the method comprising:
providing an initial population of T cells;
contacting the population of cells with a sufficient amount of a composition comprising 6-formylindolo[3,2-b]carbazole (FICZ) or beta-naphthoflavone (bNF), and
optionally evaluating the presence and/or number of T_{H}17 cells in the population;
wherein the method results in an increase in the number of regulatory T_{H}17 cells in the population.

2. The method of claim 1, wherein the initial population of T cells comprises naïve T cells or CD4⁺CD62 ligand⁺ T cells.

3. The method of claim 1, further comprising administering the T_{H}17 cells to a subject suffering from a disorder that would benefit from an enhanced T_{H}17-mediated immune response, in an amount sufficient to improve or ameliorate a symptom of the disorder.

4. The method of claim 1, wherein the population of T cells is in vitro, and the method further comprises contacting the cells with an effective amount of one or both of interleukin-6 (IL-6) and transforming growth factor (TGF)-beta.

5. The method of claim 4, further comprising preparing the enriched population for administration to a subject.

6. A method of treating a subject having a disease that would benefit from an enhanced T_{H}17-mediated immune response, the method comprising:
identifying a subject in need of treatment that would increase an immune; and
administering to the subject a composition comprising a therapeutically effective amount of 6-formylindolo[3,2-b]carbazole (FICZ) or beta-naphthoflavone (bNF),
thereby treating the subject.

7. The method of claim 6, wherein the subject is infected with a pathogen selected from the group consisting of viruses, bacteria, fungi, and protozoa.

8. The method of claim 6, wherein the subject has cancer.

9. The method of claim 1 or 6, wherein the FICZ or bNF is linked to a biocompatible nanoparticle.

10. The method of claim 1, further comprising contacting the cells with an antibody that selectively binds to an antigen present on a T cell, a B cell, a dendritic cell, or a macrophage.

11. The method of claim 10, wherein the antibody is linked to a biocompatible nanoparticle.

12. The method of claim 6, further comprising administering an antibody that selectively binds to an antigen present on a T cell, a B cell, a dendritic cell, or a macrophage.

13. The method of claim 12, wherein the antibody is linked to a biocompatible nanoparticle.

14. The method of claim 13, wherein the FICZ or bNF and antibody are colocalized on the same nanoparticles.

15. The method of claim 6, further comprising administering an antigen associated with the disease in the subject.

16. The method of claim 8, wherein the antigen is a tumor-associated antigen.

17. The method of claim 7, wherein the antigen is associated with a pathogen selected from the group consisting of viruses, bacteria, fungi, and protozoa.
